(19) United States
(12) Patent Application Publication
(10) Pub. No.: US 2020/0098480 A1
(43) Pub. Date: Mar. 26, 2020

(54) SYSTEM AND METHOD FOR PROVIDING CLINICAL OUTCOMES DRIVEN EXPERTISE FOR DISEASE TREATMENT

(71) Applicant: Mayo Foundation for Medical Education and Research, Rochester, MN (US)

(72) Inventors: Svetomir N. Markovic, Rochester, MN (US); Lisa A. Kottschade, Rochester, MN (US)

(21) Appl. No.: 16/615,264

(22) PCT Filed: May 31, 2018
(86) PCT No.: PCT/US2018/035433

§ 371 Date: Nov. 20, 2019

Related U.S. Application Data
(60) Provisional application No. 62/514,383, filed on Jun. 2, 2017.

(51) Int. Cl. G16H 50/50; G16H 50/70; G16H 70/20; G16H 10/40; G16H 10/60; G16H 20/10; G16H 15/00

(52) U.S. Cl. CPC G16H 50/50; G16H 50/70; G16H 70/20; G16H 15/00; G16H 10/60; G16H 20/10; G16H 10/40

(57) ABSTRACT

Embodiments of the disclosure include a system and method for evaluating therapeutic regimens. Embodiments of the method comprise: (1) providing an information system including a data registry component of patient records and a statistical analysis component; (2) accessing the data registry component on the basis of a patient's demographics to identify a cohort of patient records; (3) accessing the statistical analysis component to statistically analyze the identified cohort of patient records; (4) generating reports of the statistical analyses, wherein the reports include information evidencing the results of different outcomes achieved by different therapies; and (5) making the reports available for review by a physician.

### CROSS-REFERENCE TO RELATED APPLICATION

This application claims the benefit of U.S. Provisional Application 62/514,383, filed Jun. 2, 2017, which is incorporated herein by reference in its entirety for all purposes.

### BACKGROUND OF THE INVENTION

The treatment of complex disease states can be enhanced through the use of a multidisciplinary team of medical professionals. Each professional of the team contributes his or her own and unique expertise and insights developed through their clinical practice. This approach is particularly advantageous in connection with the treatment of cancers.

For example, metastatic malignant melanoma is a highly aggressive malignancy, that until recently had very limited treatment options. Recently a number of new drugs have been FDA approved for this disease. The daily task of optimally managing melanoma patients with multiple comorbidities in the face of new drug toxicities becomes increasingly complicated and dependent upon broad expertise and experience. While front-line treatments are well supported by randomized clinical trial data, management of subsequent disease progression with secondary and tertiary treatments can be optimized by an organized multidisciplinary treatment plan. National treatment guidelines rely heavily on the data from carefully controlled studies in homogenous patient populations. In contrast, however, the typical clinical practice of advanced melanoma is a longitudinal series of sequential interventions in a heterogeneous patient population.

Real world experience in managing disease states outside of clinical trials encompasses a large body of knowledge that would be useful to patient care. Unfortunately, this knowledge is largely unavailable and/or inaccessible to medical teams, especially in a timely manner. There remains a continuing need for improved systems and methods for incorporating clinical information into individualized patient treatment. Such a system and method that incorporates an extensive and deep range of clinical practice data with tools that enable the efficient and effective analysis of such data can enhance the efficacy of disease management.

### SUMMARY

Embodiments of the present invention involve the extraction and outcomes analysis of clinical data to allow a provider to make disease state management decisions, such as those relating to melanoma. In particular, embodiments allow such actions in a timely (e.g., real time) manner. Providing effective access to such a collective interdisciplinary clinical practice can rapidly yield clinical information capable of consistently enhancing treatment regime decisions.

Embodiments include a system for providing disease management through clinical outcomes driven expertise comprising a data registry component and a statistical analysis component coupled to the data registry component. Embodiments of the data registry component include one or more of: (1) patient electronic medical records (EMRs) within one or more health care provider systems (e.g., maintained in a unified data platform (UDP)); (2) clinical notes made by medical personnel (e.g., and included in patients' EMRs or other records); (3) patient laboratory test results (e.g., maintained in a laboratory record and information system); (4) other patient information repositories such as a clinical documents manager (CDM) where other patient treatment regimens are maintained; (5) sources of published medical guidelines and the results of relevant clinical studies; (6) diagnosis code records (e.g., identifying patients with specific disease states and patients that received treatment from a specific health care provider); (7) patient and associated demographic information (e.g., gender, age, ethnicity, marital status, vital status); (8) first line, second line, third line and/or forth line treatment regimens (e.g., therapy and immunotherapy drugs, doses and associated start/end dates); (9) outcomes or results associated with treatment regimens (e.g., tumor burden status, incidence of symptomatic metastasis, incidence of brain metastasis); (10) lab data such as patient genomics (e.g., status of BRAF, cKit, NRAS, GNAQ, GNA11); and (11) lab data such as patient markers (e.g., LDH level). Embodiments of the statistical analysis component are coupled to the data registry component and comprise graphical user interfaces and tools including one or more of: (1) a cohort builder tool enabling a physician to define custom cohorts to identify patient populations of interest; (2) a cohort analyzer tool supporting advanced cohort analysis, including the development of optimal, patient-specific treatment plans; (3) a time to treatment change tool to model time to treatment changes across various therapeutic agents as proxies to determine responses to therapy; (4) an overall survival tool to model overall survivals; (5) a total time on treatment tool; and (6) a therapeutic sequence tool to model the impacts of therapeutic sequences on time to treatment changes and inform how drug regimens are administered for patients.

Embodiments further include a data extraction component coupled to the registry component and the statistical analysis component and operable to identify, define and/or locate important data elements in the data registry component. The data registry component can comprise a structured data registry.

Embodiments of the system are configured for melanoma management.

Embodiments also include a method comprising: (1) providing an information system including a data registry component of patient records and a statistical analysis component; (2) accessing the data registry component on the basis of a patient's demographics to identify a cohort of patient records; (3) accessing the statistical analysis component to statistically analyze the identified cohort of patient records; (4) generating reports of the statistical analyses, wherein the reports include information evidencing the results of different outcomes achieved by different therapies; and (5) making the reports available for review by a physician.

Accessing the statistical analysis component to statistically analyze the patient records includes one or more of analyzing time to treatment change, overall survival, total time on treatment or therapeutic sequence in embodiments.

In embodiments, accessing the data registry component further includes accessing the data registry component on the basis of the patient's first line drug regimen/therapy; and generating reports of the statistical analyses includes generating reports including information evidencing the results of different outcomes achieved by second and/or subsequent line therapies following first line therapies using the first line drug regimen/therapy.

Accessing the data registry component further includes accessing the data registry component on the basis of clinical characteristics, optionally tumor burden and/or symptomatic mets, associated with the patient, in embodiments.

Embodiments of the method are configured for treating melanoma.

In embodiments, accessing the statistical analysis component can include selecting cohorts and selecting drugs. Selecting drugs can include selecting a line of therapy and selecting a plurality of drugs in embodiments.

### BRIEF DESCRIPTION OF THE DRAWINGS

FIG. 1 is block diagram of components of a system in accordance with embodiments of the invention.

FIG. 2 is diagrammatic illustration of a computer system in accordance with embodiments that can be used to implement the system shown in FIG. 1.

FIG. 3 is block diagram illustration of embodiments of the statistical analysis component shown in FIG. 1.

FIG. 4 is an illustration of embodiments of a graphical user interface and display that can be used by the physician with the cohort builder tool shown in FIG. 3.

FIG. 5 is an illustration of embodiments of a graphical user interface and display that can be used by the physician with the cohort analyzer tool shown in FIG. 3.

FIGS. 6A and 6B are illustrations of embodiments of displays that show tabulated and graphed time to treatment change information for different cohorts in connection with the time to treatment change tool shown in FIG. 3.

FIGS. 7A and 7B are illustrations of embodiments of displays showing tabulated and graphed overall survival information for different cohorts in connection with the overall survival tool shown in FIG. 3.

FIG. 8 illustrates embodiments of a display showing tabulated therapeutic sequence for several lines of therapy in connection with the therapeutic sequence tool shown in FIG. 3.

### DESCRIPTION OF THE INVENTION

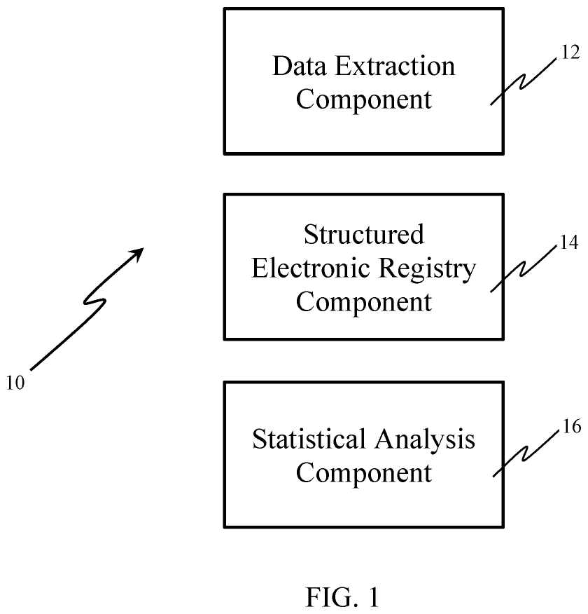

A system 10 for enabling disease management using clinical outcomes driven expertise in accordance with embodiments of the invention is illustrated generally in FIG. 1. As shown, system 10 includes data extraction component 12, structured electronic registry component 14 and statistical analysis component 16. Although described below in connection with melanoma for purposes of example, other embodiments of system 10 can be similarly configured for use in connection with other disease states or disorders such as other cancers, cardiovascular disease, etc.

Data extraction component 12 operates to identify, define and locate critical or important data elements that influence a physician's clinical decision-making. Examples of the sources of information can that can be used and processed by the data extraction component 12 include:
Patient electronic medical records (EMRs) within one or more health care provider systems (e.g., maintained in a unified data platform (UDP))
Clinical notes made by medical personnel (e.g., and included in patients' EMRs or other records)
Patient laboratory test results (e.g., maintained in a laboratory record and information system)
Other patient information repositories such as a clinical documents manager (CDM) where other patient treatment regimens are maintained)
Sources of medical literature, randomized medical trials and published medical guidelines

The EMRs and clinical notes can be compilations of information associated with patients, such as information from the patients' general practitioners, specialists (e.g., radiologist notes) and laboratory results. Tools such as natural language processing (NLP) and machine-assisted human abstraction (MAHA) can be incorporated into the data extraction component 12 and used to obtain relevant information from the information sources. For example, information that might otherwise have been implied within existing data can be extracted by MAHA and annotated as discrete data in an EMR.

Electronic registry component 14 is a database of information such as information provided directly from information sources such as those described above, and/or provided by the data extraction component 12. In embodiments, registry component 12 structures the stored data in a manner that enhances subsequent analyses of the data by statistical analysis component 16. Much, although not all, of the information maintained in the registry component 14 can be specific to patients for a specific diagnosis such as melanoma and linked to a specified therapeutic intervention. Examples of the types of information maintained in the registry component 14 include:
Diagnosis code records (e.g., identifying patients with specific disease states and patients that received treatment from a specific health care provider)
Patient and associated demographic information (e.g., gender, age, ethnicity, marital status, vital status)
First line, second line, third line and forth line treatment regimens (e.g., therapy and immunotherapy drugs, doses and associated start/end dates)
Outcomes or results associated with treatment regimens (e.g., tumor burden status, incidence of symptomatic metastasis, incidence of brain metastasis)
Lab data such as patient genomics (e.g., status of BRAF, cKit, NRAS, GNAQ, GNA11)
Lab data such as relevant tumor markers (e.g., LDH level)

Statistical analysis component 16 allows physicians to select specific patient cohorts from the electronic registry component 14, and performs specified analyses on the associated clinical data to validate and provide an understanding of the implications of treating patients with a complex set of shared characteristics. The information generated by analysis component 16 can be presented to physicians in a manner that enables the physicians to evaluate the efficacy of different therapies under different circumstances.

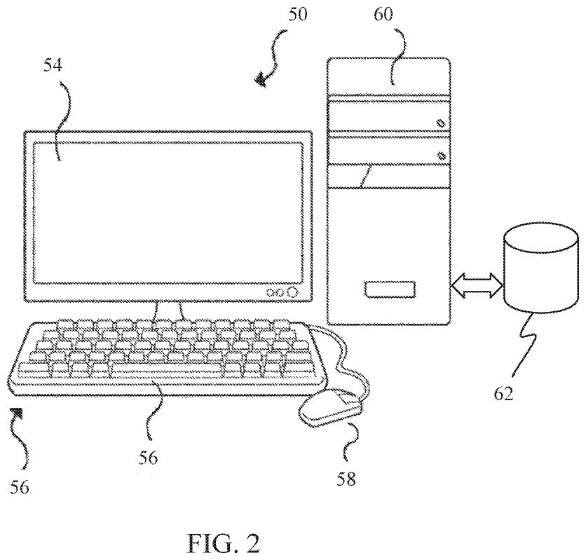

FIG. 2 is a diagrammatic illustration of a computer system 50 implementing the system 10 in accordance with embodiments of the invention. As shown, computer system 50 includes a graphical user interface 52 having a monitor 54, keyboard 56 and mouse 58. A processing system 60 is coupled to the user interface 52 and to database 62. Database 62 is represented diagrammatically in FIG. 1, and can take any known or otherwise conventional logical and/or physical (e.g., local or distributed) form. Computer system 50 can be interfaced (e.g., by wireless or wired networks, not shown) to other computer or data collection systems (not shown) such as a health care providers EMR and laboratory management systems (which can form at least part of the database 62 in embodiments). Conventional software programs and algorithms for performing NLP and MAHA, as well as other features of data extraction component 12, can be stored in the database 62 and used by the processing system 60. For example, data elements identified, defined and/or located by data extraction component 12 can be stored in the database 62. Database 62 can maintain the structured electronic registry component 14, as well as programs used by statistical analysis component 16. User interface 52, including graphical user interfaces, can be used by a physician to interact with the system 10 (e.g., to select cohorts of patients' statistical analysis tools). The information generated by the statistical analysis component 16 can be presented to the user on monitor 54, for example. The illustrated embodiment of computer system 50 is shown for purposes of example, and other embodiments of the invention have different or additional components, such as other user interfaces for administrators and providers that use the system, and different or additional memory and database structures.

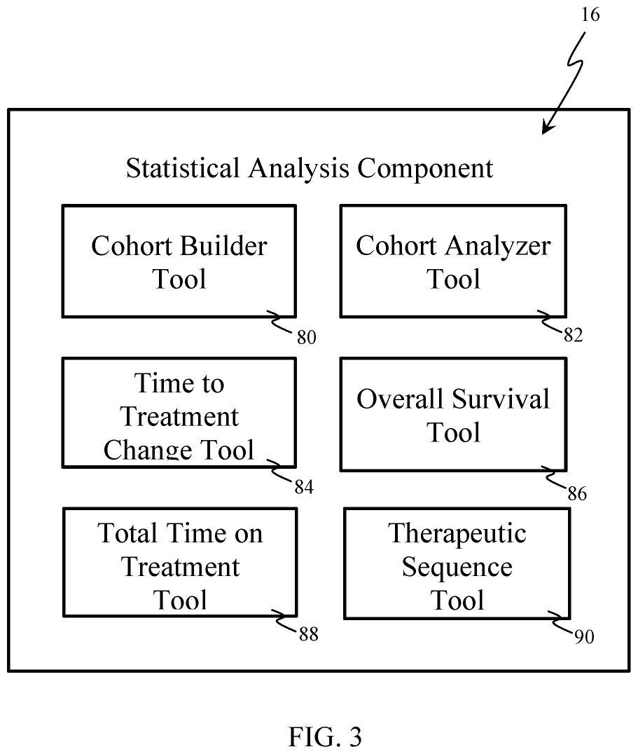

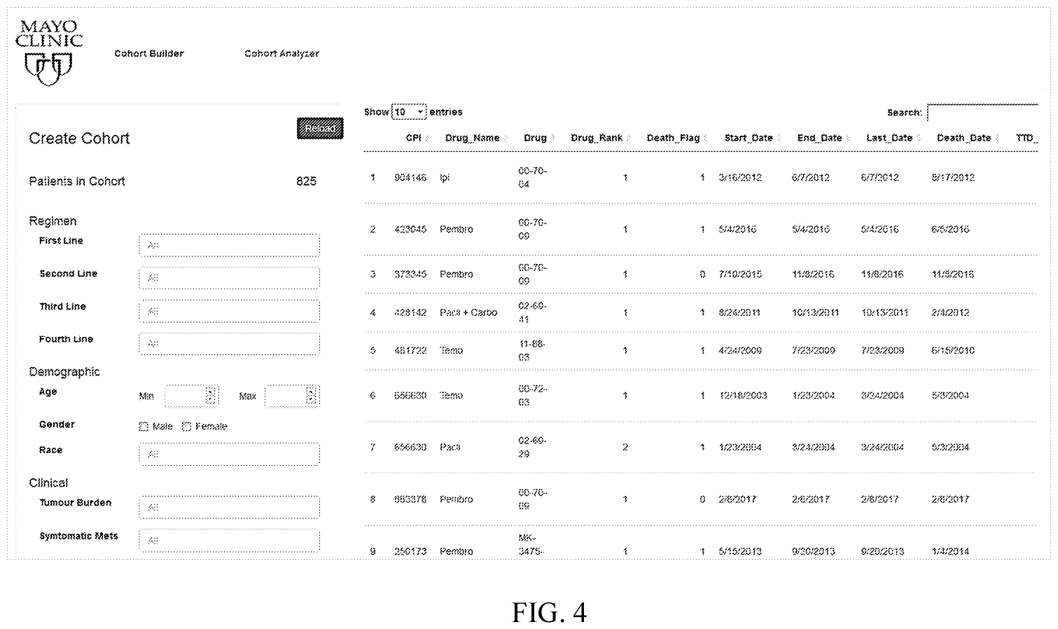

The operation of statistical analysis component 16 can be described in greater detail with reference to FIGS. 3-5, 6A, 6B, 7A, 7B and 8. As shown in FIG. 3, statistical analysis component 16 includes a number of tools that can be used by a physician. The tools include cohort builder tool 80, cohort analyzer tool 82, time to treatment change tool 84, overall survival tool 86, total time on treatment tool 88 and therapeutic sequence tool 90. The tools 80, 82, 84, 86, 88 and 90 can be selected using the graphical user interface available to the physician through the computer system 50. Using the cohort builder tool 80, a physician can create or identify cohorts of patients from electronic registry component 14 having specific characteristics selected by the physician (e.g., demographic information corresponding to a patient under the care of the physician). FIG. 4 is an illustration of a graphical user interface and display that can be used by the physician with cohort builder tool 80. As shown, the interface includes drop down or other menus that allow the physician to select specific patient characteristics such as a specific line of treatment, demographics, and comorbidities. Cohort builder tool 100 identifies patient records meeting the selected patient characteristics, and provides a display of pertinent portions of the records such as the drugs and drug rank. Cohorts created by the physician using the tool 100 can be saved for subsequent use by the statistical analysis component 16.

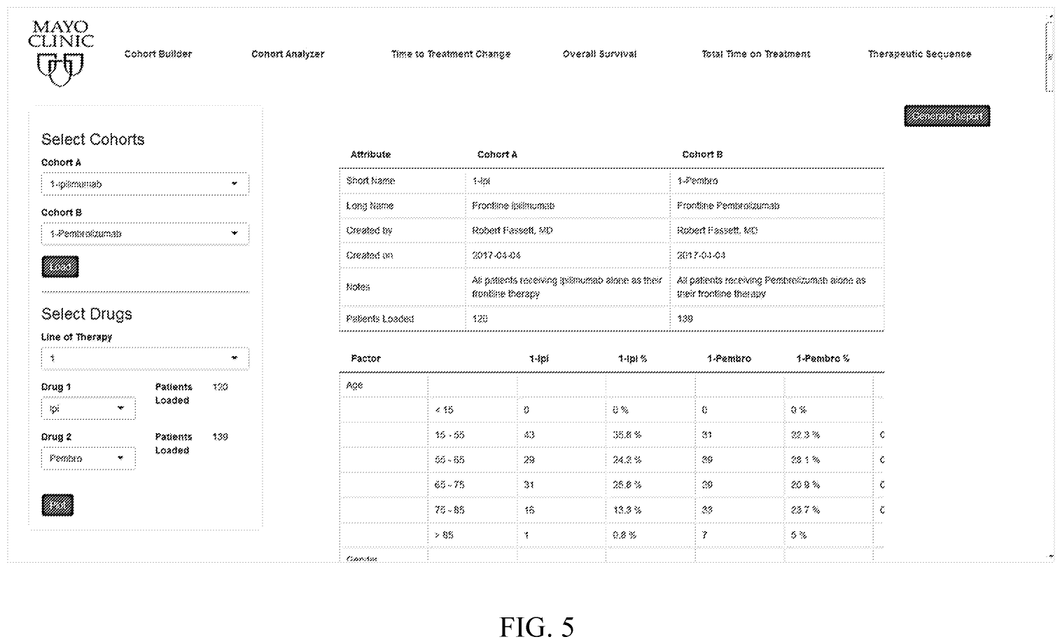

Cohort analyzer tool 82 can be used to perform statistical analyses on the cohorts created using the tool 100. Cohort analyzer tool 82 supports advanced cohort analysis, and enables the development of optimal, patient-specific treatment plans. FIG. 5 is an illustration of a graphical user interface and display that can be used by the physician with cohort analyzer tool 82. As shown, the interface allows a physician to select one or more previously identified and stored cohorts, as well as the line of therapy and drugs used by the patients in those cohorts. Attributes of the selected cohorts, as well as factors of the patients indexed by characteristics such as age and gender can be displayed.

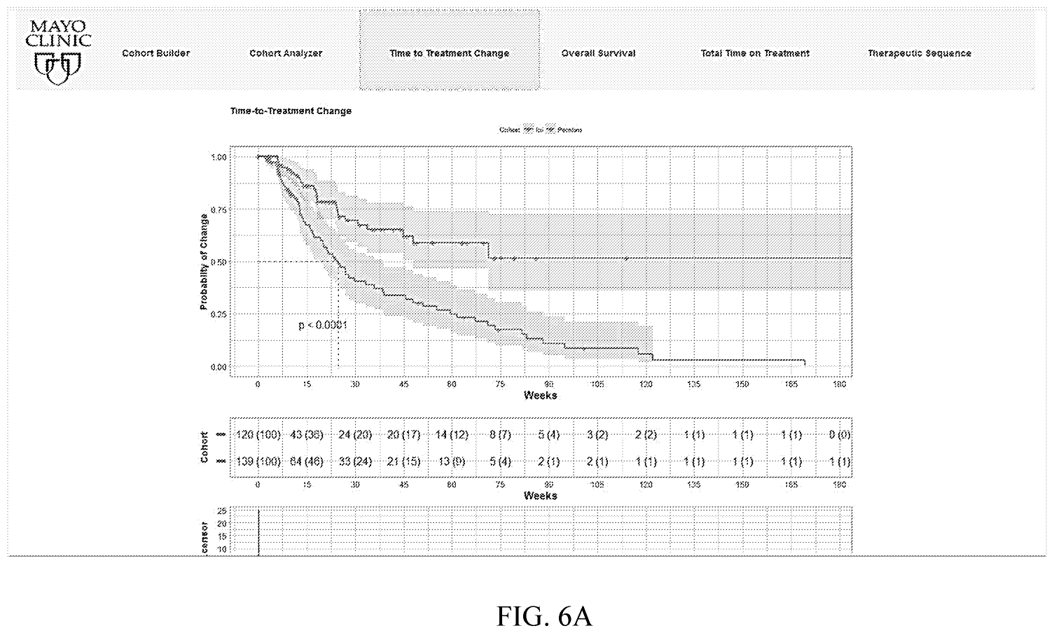

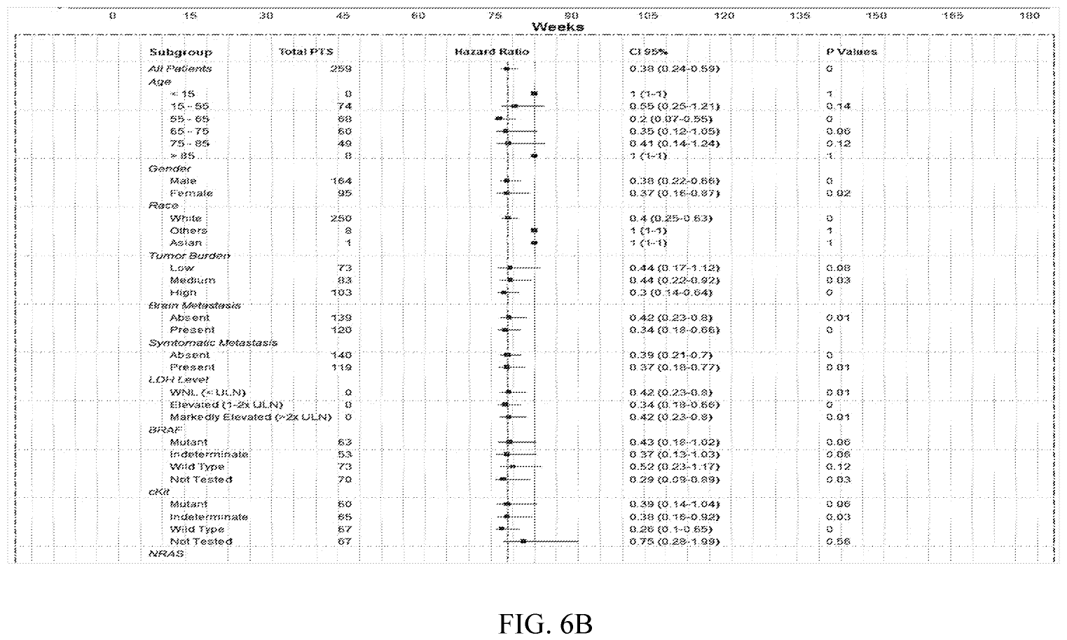

Time to treatment change tool 84 can be used to model and display time to treatment changes for the selected cohorts. The displayed information can be used by the physician to analyze treatment changes across various therapeutic agents as a proxy to determine responses to therapy and/or toxicity to therapy. FIGS. 6A and 6B are illustrations of displays 120 that show tabulated and graphed time to treatment change information for the different cohorts.

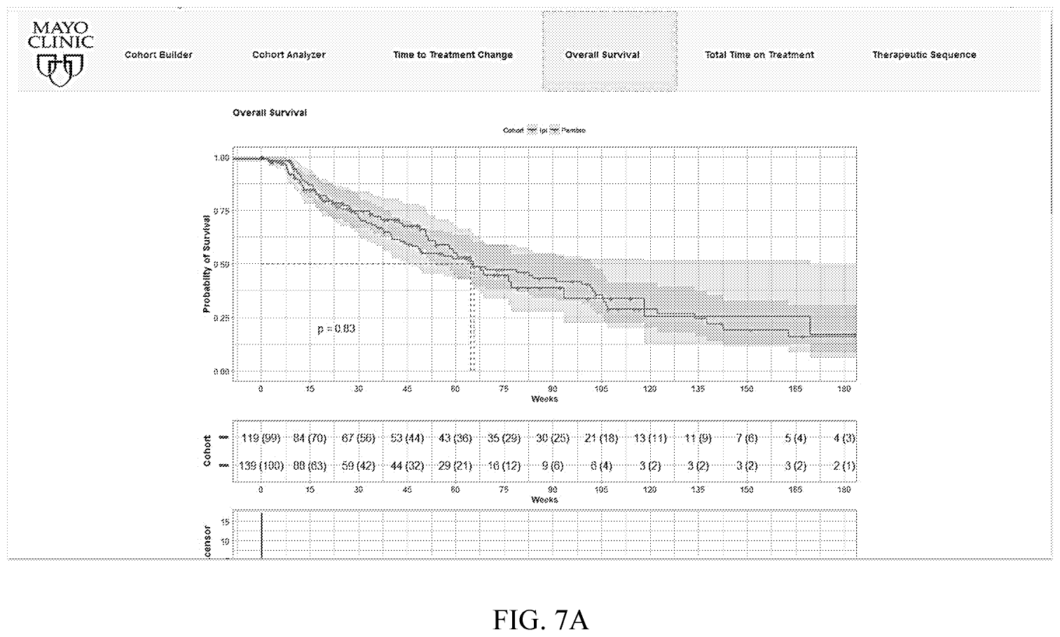

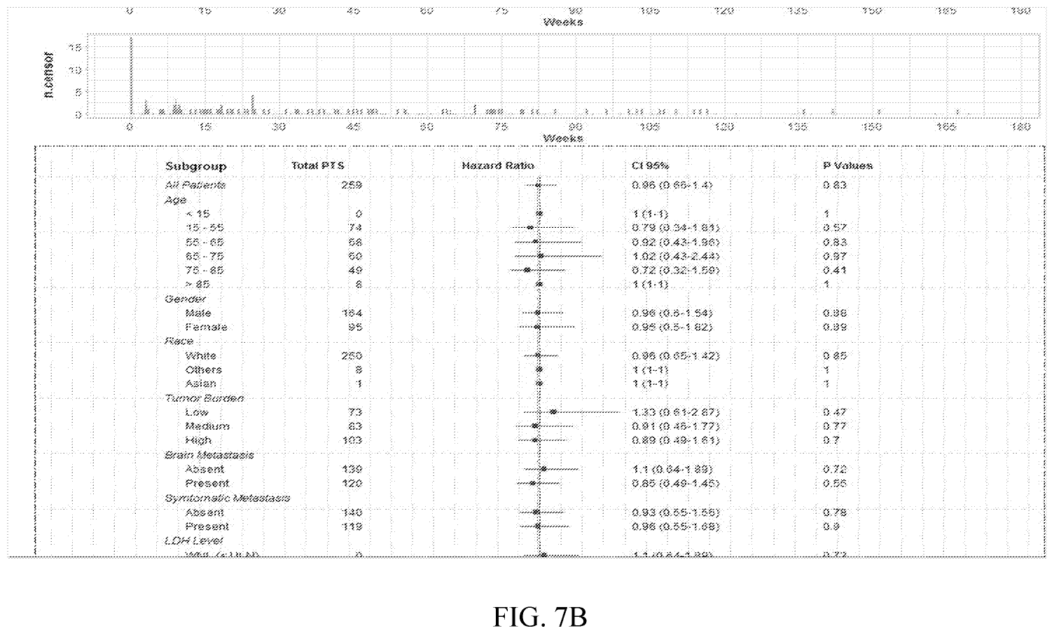

Overall survival tool 86 can be used to model and display overall survival rates for the selected cohorts. The overall survival rate information can be used by the physician to analyze survival rates as a proxy to determine responses to therapy. FIGS. 7A and 7B are illustrations of displays showing tabulated and graphed overall survival information for different cohorts in accordance with embodiments.

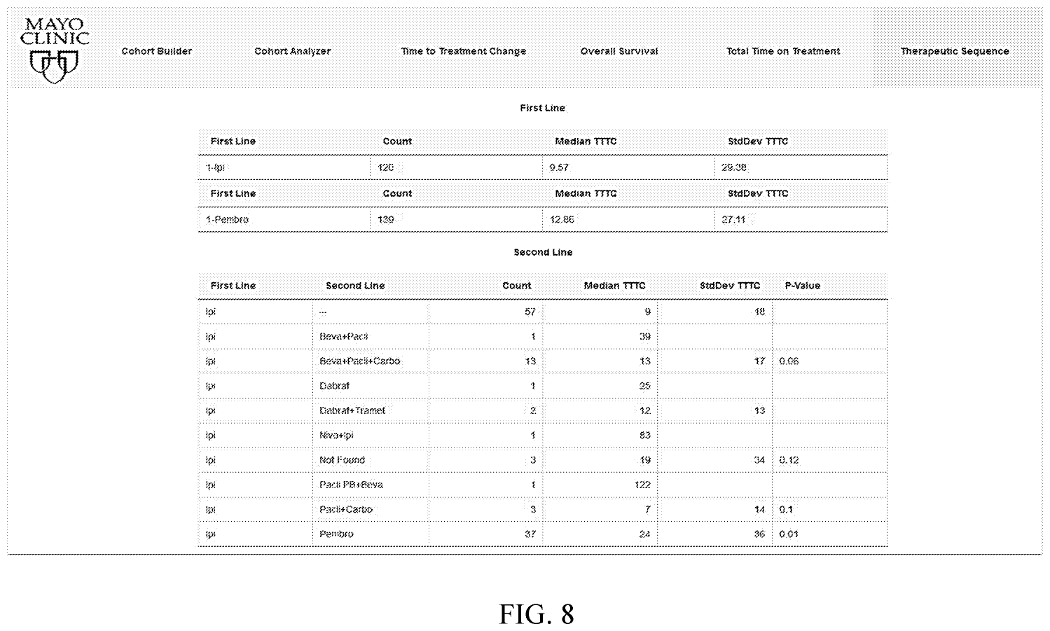

Therapeutic sequence tool 90 can be used to provide information on the different lines of therapy provided to the selected cohorts. This information can be used by physicians to understand the impact of therapeutic sequences on time to treatment change (TTTC), thereby informing how drug regimens are administered for each patient with a certain set of characteristics. FIG. 8 illustrates a display showing tabulated therapeutic sequence for several lines of therapy in accordance with embodiments.

Embodiments of the invention offer important advantages. For example, they can assist a physician in offering data-driven, personalized clinical treatment regimens. Large bodies of knowledge can be effectively and efficiently used, thereby emulating results obtained through multidisciplinary approaches. Physicians are able to evaluate potential clinical outcomes based on unique treatments and other variables across patient cohorts with shared characteristics. Physicians are also able to adapt treatment strategies as patients respond to a given regimen. Optimal treatment plans can be identified. The result is a substantial enhancement of patient care.

As use case examples, a physician treating patients for metastatic melanoma can prescribe as first line treatments a drug regimen/therapy that demonstrated efficacious results in randomized clinical trials on patients presenting similar symptoms. Alternatively, the physician can prescribe as a first line treatment a drug regimen/therapy that the physician self-determined based on his or her clinical experience of success with patients presenting similar symptoms. In both examples, post-therapy reviews of the patients (e.g., through imaging or lab analyses) indicate a positive (e.g., complete tumor) response to the first drug regimen/therapy. The physician therefore decides that it is appropriate to keep the patients on the first or a modified version of the first drug regime/therapy.

However, at a time following the post-therapy reviews, the patients present themselves with evidence of progression of the disease, and reviews of the patients confirm progression of the disease. The physician must therefore determine how best to treat the disease progression in these patients. In these situations, recommendations and outcomes of leading clinical trials are not suitable for the best course of treatments. Typically, a physician may rely upon his or her personal clinical experience, and/or consult with colleagues, to determine whether to discontinue or modify the first drug regimen/therapy, or to identify an alternative (e.g. second line) drug regimen therapy.

Using embodiments of the system 10 and method of the invention, the physician can evaluate alternative therapies and identify patient-specific next line therapies that have been demonstrated by clinical practice to provide efficacious results. For example, using the cohort builder tool 80, the physician can identify cohorts of patients in the electronic registry component 14 having similar demographics and clinical outcomes following treatment using a similar first line drug regimen/therapy. Analyses of these cohorts can then be performed using tools such as 82, 84, 86, 88 and 90, providing the physician with reports of information of the results of different outcomes achieved by different therapies. Based on a review of the reports, the physician can make data-driven decisions on appropriate second line drug regimens/therapies for each individual patient. Each patient is treated based on their unique characteristics and situations, thereby enhancing of an efficacious treatment plan. This method and the use of embodiments of the invention can be repeated for third and subsequent line therapies (e.g., if the patient does not adequately respond to the previous therapy or has a toxicity from the treatment).

Although the invention has been described with reference to preferred embodiments, those of skill in the art will recognize that changes can be made in form and detail without departing from the spirit and scope of the invention.

1. A system for providing disease management through clinical outcomes driven expertise, comprising:
a data registry component including one or more of:
patient electronic medical records within one or more health care provider systems;
clinical notes made by medical personnel;
patient laboratory test results;
other patient information repositories such as a clinical documents manager where other patient treatment regimens are maintained;
sources of published medical guidelines and the results of relevant clinical studies;
diagnosis code records;
patient and associated demographic information;
first line, second line, third line and/or forth line treatment regimens;
outcomes or results associated with treatment regimens;
lab data such as patient genomics; and
lab data such as patient markers; and
a statistical analysis component coupled to the data registry component, the statistical analysis component including graphical user interfaces and tools including one or more of:
a cohort builder tool enabling a physician to define custom cohorts to identify patient populations of interest;
a cohort analyzer tool supporting advanced cohort analysis, including the development of optimal, patient-specific treatment plans;
a time to treatment change tool to model time to treatment changes across various therapeutic agents as proxies to determine responses to therapy;
an overall survival tool to model overall survivals;
a total time on treatment tool; and
a therapeutic sequence tool to model the impacts of therapeutic sequences on time to treatment changes and inform how drug regimens are administered for patients.

2. The system of claim 1 and further including a data extraction component coupled to the registry component and the statistical analysis component and operable to identify, define and/or locate important data elements in the data registry component.

3. The system of claim 1 wherein the data registry component comprises a structured data registry.

4. The system of claim 1 configured for melanoma management.

5. A method, comprising:
providing an information system including a data registry component of patient records and a statistical analysis component;
accessing the data registry component on the basis of a patient's demographics to identify a cohort of patient records;
accessing the statistical analysis component to statistically analyze the identified cohort of patient records;
generating reports of the statistical analyses, wherein the reports include information evidencing the results of different outcomes achieved by different therapies; and
making the reports available for review by a physician.

6. The method of claim 5 wherein accessing the statistical analysis component to statistically analyze the patient records includes one or more of analyzing time to treatment change, overall survival, total time on treatment or therapeutic sequence.

7. The method of claim 6 wherein:
accessing the data registry component further includes accessing the data registry component on the basis of the patient's first line drug regimen/therapy; and
generating reports of the statistical analyses includes generating reports including information evidencing the results of different outcomes achieved by second and/or subsequent line therapies following first line therapies using the first line drug regimen/therapy.

8. The method of claim 7 wherein accessing the data registry component further includes accessing the data registry component on the basis of clinical characteristics, optionally tumor burden and/or symptomatic mets, associated with the patient.

9. The method of claim 8 for treating melanoma.

10. The method of claim 5 wherein accessing the statistical analysis component includes selecting cohorts and selecting drugs.

11. The method of claim 10 wherein selecting drugs includes selecting a line of therapy and selecting a plurality of drugs.
